(19) United States
(12) Patent Application Publication
(10) Pub. No.: US 2004/0258779 A1
(43) Pub. Date: Dec. 23, 2004

(54) Dietary and pharmaceutical compositions for treatment of anemia, thrombocytopenia, and leukopenia

(76) Inventors: Boris V. Pinegrin, Moscow (RU); Todor Dimitrov, Chestnut Hill, MA (US); Vyacheslav L. Lvov, Moscow (RU); Vladimir I. Slesarev, Boyds, MD (US); Musa R. Khaitov, Moscow (RU)

Correspondence Address: Vladimir Slesarev, 18406 Broad Leaf Rd., Boyds, MD, 20841, US

(21) Appl. No.: 10/455,123

(22) Filed: Jun. 6, 2003

(51) Int. Cl. A61K 35/78

(52) U.S. Cl. 424/757

(57) ABSTRACT

A novel peptidoglycan compositions are offered for inhibition of apoptosis of hematopoietic cells. The active ingredient is N-acetyl-glucosamine-N-muramyl L-alanine-D-isoglutamine-meso-diaminopimelyl-D-alanine (GMTP). Its cytoprotective effect allows to treat leucopenia, anemia, and thrombocytopenia.

### FIELD OF INVENTION

The present invention relates to apoptosis modulating glucosamine-muramyl-peptides, obtained by specific endopeptidase digestion of gram positive and gram negative bacteria, methods of preparation thereof, medical food compositions and methods of using them in dietary management and treatment of anemia, leucopenia, and thrombocytopenia.

### BACKGROUND OF THE INVENTION

The formation of blood cells is regulated by colony-stimulating factors (CSF's), which promote colony formation and proliferation of different cells, and by the factors, which stimulates differentiation and maturation. Many of them affect more than single cell lineage. For example, the stimulation of both platelet and erythrocyte production is caused by erythropoietin (EPO). Thrombopoietin (TPO) possesses both megakaryocyte-CSF (Meg-CSF) and megakariocyte potentiator activity in the development of megakaryocytes. Moreover, additional factors are involved in the development of any given cell lineage. Thus, MEG-SCF include interleukin-3 (IL-3), granulocyte-macrophage colony-stimulating factor (GM-CSF) and stem cell factor, and Megakaryocyte potentiating factor reputedly include IL-6,IL-7,IL-11, EPO and leukemia inhibitory factor.

Chemotherapeutic agents blunt EPO response. Cisplatin is particularly toxic, causing a prolonged anemia (Wood P A and Hrushesky W J, J Clin Invest, 1995, 95:1650). Cyclosporine A also attenuates the production of EPO and this may contribute to the anemia of patients undergoing organ transplantation. (Schrezenmeier H, et al., Br. J. Hematol., 1994, 88:286). Finally, a direct suppression of EPO formation by human immunodeficiency virus has been observed in vitro, suggesting that this may have a role in the pathogenesis of HIV anemia (Wang Z., et al., Exp. Hematol., 1993, 21:683).

Anaplastic anemia (AA) can be the result of failure to control the virus. It was demonstrated that mice devoid of Tumor Necrosis Factor (TNF) alpha receptor (R) 1 died from anaplastic anemia caused by lymphocytic choriomeningitis virus (LCMV), whereas the mice with this TNFR1 have survived and became virus carries (Binder D., et al., J. Exp.Med., 1998, 187:1903-1920). The injection of TNF into rodents may produce a stimulatory effect on hematopoesis by induction of colony-stimulating activities in the endothelium. The results in LCMV-infected mice rather support the interpretation that the ultimate effect of TNF alpha in virus associated anaplastic anemia is suppressive via induction of apoptosis of bone morrow progenitors and is not stimulatory (Johnson C S, et al., Blood, 1988,72:1875-1883).

In humans pancytopenia clinically presents gradually, usually 1-7 week after episodes of acute hepatitis. The experiments suggest that progression of anaplastic anemia and functional exhaustion of virus-specific CD8 T cells are ruled by distinct pathogenetic mechanisms during persistent virus infection. Persistent expression of viral antigen in stromal and dendritic cells of bone marrow sustains local infiltration with CD8+ T cells and causes anaplastic anemia via bystander destruction of blood cell precursors by T cell-secreted cytokines, predominantly TNF alpha.

Absence of one toxic CD8+ T cell dependent cytokine such as TNF alpha shifts this lethal balance and rescue bone morrow. This mechanism of peripheral tolerance induction may explain why rare patients survive anaplastic anemia and remission to normal blood cell values can “naturally” occur (Young, N S and Alter B P, Aplastic anemia:acquired and inherited, 1994).

Anemia of chronic disease (ACD) is associated with cancer, rheumathoid arthritis, multiple myeloma, non-Hodgkin lymphoma, myelodysplastic syndromes, idiopatic myelofibrosis, and end-stage renal disease (Stenvinkel P. Nephrol.Dial.Transplant., 2001,16: 36-40). TNF alpha, interferon gamma, interleukine 6 and interleukine 1 beta are produced in the bone marrow or other organs of patients with this disease. These cytokines have been implicated in the pathogenesis of ACD because they inhibit erythropoesis while fostering the development and function of marrow cells involved in inflammation. A final common pathway for the inhibition is likely to be the induction of erythroid cell apoptosis. (Hellestrom-Lindberghell Clinical evidence for the relation between TNF alpha and ACD comes from studies of monoclonal antibodies to TNF alpha, which ameliorate signs and symptoms in chronic inflammatory disease. Rheumatoid arthritis patients, who were treated with monoclonal antibodies to TNF alpha showed improvement in their anemia that was not mediated through changes in erythropoietin.(Papadaki H., et al., Blood, 2002, 100: 474-482).

In parallel, TNF alpha plays a critical role in the control of neutrophil survival by inducing an apoptotic death program which can be rapidly triggered by a variety of stimuli. When neutrophils were pretreated with TNF alpha and then were exposed to different inflammatory agents, there was a marked stimulation of apoptosis. A broad panel of stimuli which includes cytokines IFN gamma and GM-CSF was found to make a difference in triggering apoptosis of neutrophils treated with TNF alpha. By contrast, a slight increase in the number of apoptotic cells was also found, when neutrophils were cultured only with TNF alpha (Salamone G., et.al, J.Immunol.,2001,166:3476-3483)

Dysplasia of megakaryocytic, granulocytic, and erythroid lineages are the hallmarks of myelodysplastic syndromes. The apoptosis prevails kinetically over increased proliferation, causing the peripheral cytopenia. In MDS many studies link overexpression of TNF alpha to cell death (Head D R., et al., Leukemia, 1996, 10:1826, Lancet J E., et al., Hematol/Oncol.Clin.N.Am., 2000,14:251-267). TNF alpha produced by MDS mononuclear cells is inhibitory to both normal and MDS colony growth indicating that residual normal hematopoiesis can also be blocked in MDS (Head D R., et al., Leukemia, 1996, 10:1826). The identification of TNF alpha as a key cytokine in cell death regulation and increased susceptibility of MDS cells to TNF alpha is the basis for several clinical trials of TNF-alpha inhibitors (Bennett J M, et al., Br.J.Haematol., 1982, 51:189-199).

Immunosupressive treatment which includes prednisone, antithymocyte globulin, cyclosporine, thalidomide, chemotherapy, and soluble TNF alpha receptor is currently in use for treatment of MDS. Amifostine, Pentoxifylline, and Ciprofloxacin are offered for cytokine inhibition (List A F, et al., Blood, 1997, 90:3364-3369, Raza A, et al., Blood, 2000,95:1580-1587).

A number of drugs for treatment of thrombocytopenia are currently in the studies. These include recombinant trombopoietin, pegylated recombinant human megakaryocyte growth and development factor, lisofylline (Clark et al., Cancer Res., 1996,56:105-112), recombinant IL-1, recombinant IL-3, recombinant IL-6, recombinant IL-1, and recombinant G-CSF, among others. Certain of these experimental treatments for thrombocytopenia appear to be limited by lack of efficacy, toxicity, or both. For example, treatment with recombinant IL-3 has been disappointing in terms of supporting platelet and granulocyte production and complicated by unacceptable toxicity. Similarly, treatment with recombinant IL-6 has been disappointing in terms of supporting platelet production and is also limited by toxicity.

In recent years, three recombinant human hemopoietic growth factors became available for clinical use: EPO for treatment of anemia, and granulocyte colony-stimulating factor and granulocyte-monocyte colony stimulating factor for treatment neutropenia. While these factors have proven to be generally safe and effective, they are expensive and still can cause side effects such as depletion of platelets in case of G-CSF administration.

Nevertheless, other hematopoietic growth factors and cytokines, including TPO and IL-3, IL-6, and IL-11 are under development and/or study as potential hematopoietic agents. In view of the forgoing, a need still exist to develop methods and compositions for treating and/or preventing anemia, thrombocytopenia, and neutropenia.

The present invention is based on the discovery that cell wall fragment of both gram negative and gram positive bacteria N-acetyl-glucosamine-N-acetyl-muramyl-L-alanine-D-isoglutamine-diaminopimenic acid-D-alanine inhibits TNF alpha mediated cytotoxicity. In parallel, this disaccharide tetrapeptide stimulates of TNF alpha and GM-CSF synthesis. Consequently, in one aspect the invention provides new glucosamine muramyl tetrapetide containing food composition, which demonstrates the modulation of TNF alpha mediated apoptosis. This modulation of TNF alpha killing pathways proved to be clinically effective in the management anaplastic anemia, netropenia, and thrombocytopenia. Concurrent stimulation of TNF alpha is useful in preventing cancer, leukemia, and sepsis. Applicants also propose peptidoglycans compositions of L. Plantarum, which enhance the red blood cells, white blood cell, and platelets count in patients with anaplastic anemia.

Another aspect of the present invention is to provide a novel medical food consisting of the probiotic peptidoglycans containing N-acetyl-glucosamine-N-Acetyl-muramyl-L-ananyl-D-isoglutamyl-meso-pilmenyl-D-alanine and soy isoflavones. This food ingredients posses synergistic effect on the inhibition of TNF alpha cytotoxicity. Such medical food may be used to reduce cancer and hepatitis associated leuekopenia and thrombocytopenia. Specifically, the present food may be recommended for those patients who suffer from common postchemotherapy toxicity such as leukocytopenia, thrombocytopenia, elevated free iron and high bilirubin with elevated liver enzymes. Further, the present invention provides nutrition for dietary management of leucopenia, cancer cachexia and muscle dystrophia.

In a related aspect, the present invention provides a food useful for treating patients suffering from myeilodysplastic syndrome. Probiotic disaccharide-tetrapeptide fortified food and drink may be especially beneficial for people with concurrent liver cirrhosis, thus preventing severe fatigue and brain damage caused by ammonia. Furthermore, presented invention provides the food for metabolic detoxifications of the carcinogenic chemicals and mutagens, which lead to anemia.

Another aspect of the present invention includes nutritional methods for the management of anemia in patients with rheumatoid arthritis. Therapeutic effect is based on newly discovered phenomena of the inhibitory effects of probiotic peptidoglycans over TNF-alpha cytotoxicity. This inhibition does not lead to reducing TNF alpha level in the blood, thus eliminating the risk of septic complications and cancer. Moreover, such effects are beneficial for rheumatoid arthritis because smoothers cytotoxic TNF alpha effects, which play a crucial role in the pathogenesis of this disease.

Still another aspect of the present invention includes dietary methods of inhibiting anemia caused by ribovarin in the patients with hepatitis C.

While another aspect of this invention is to provide a method to treat or prevent anemia, thrombocytopenia, or neutropenia by administering to a subject having or at risk of developing anemia, thrombocytopenia, or neutropenia a combination a dietary peptidoglycans of L. Plantarum as medical food and anemia, thrombocytopenia, or neutropenia medicament.

In certain embodiments, peptidoglycan is derived from gram negative E. Coli and the anemia medicament is selected from the group consisting of recombinant EPO, recombinant GM-CSF, recombinant G-CSF, recombinant IL-11, ferrous iron, ferric iron, vitamin B12, vitamin B6, vitamin C, folate, vitamin D, calcitriol, alphacalcidol, androgen, and carnitine. In a preferred embodiment the anemia medicament is recombinant EPO.

In certain embodiments, the thrombocytopenia medicament is selected from the group consisting of glucocorticoid, recombinant TPO, recombinant MGDF, pegylated recombinant MGDF, lisophylline, recombinant IL-1, recombinant IL-3, recombinant IL-6, and recombinant IL-11.

Yet another aspect of this invention comprises oral administration of the probiotic glucoproteins and/or disasshride tetrapeptide of E. coli to treat neutropenia in combination with G-CSF, recombinant GM-CSF, recombinant IL-6, glucocorticoid, recombinant IL-3,recombinant IL-1, androgens, recombinant IFN-gamma, small molecule G-CSF mimetics, G-CSF receptor antagonists, and uteroferrin. In a preffered embodiment the neutropenia medicament is recombinant G-CSF. Daily peptidoglycan dosage in the range from 200 mg to 2000 mg may be found to be acceptable for dietary management of anemia, thrombocytopenia, and neutropenia with optimal range of 1000-1500 mg per day. Daily isolated disaccharide tetrapeptide dosage in the range of from 5 mg to 100 mg would acceptable with optimal range 10-50 mg.

Still, another aspect of this invention covers newly discovered phenomena of the inhibitory effects of soy isoflavones over TNF alpha cytotoxicity. A retained natural level in the range 50-70 mg of isoflavones may be found suitable for such dietary management. A daily dose of 50-75 g of isolated soy proteins with at least 0.1 weight percent of the retained isoflavones is preferable serving quantity.

### BRIEF DESCRIPTION OF THE DRAWINGS

For further details, reference is made to the discussion which follows, in light of the accompanying drawings, wherein:

FIG. 1 illustrates the inhibition of lactate degedrogenase (LDH) release by peptidoglycans of L. Plantarum.

FIG. 2 demonstrates the synergistic effect of soy isoflavones and peptidoglycans of L. Plantarum on LDH release.

### DETAILED DESCRIPTION OF THE INVENTION

The present invention relates to the dietary and pharmaceutical inhibition of TNF alpha cytotoxicity and food and drinks, containing, as an effective component N-Acetyl-glucosamine-N-acetyl-muramyl-L-alanine-D-isoglutamine, meso-diaminopimelic acid, D-alanine-peptidoglycan extracted from E. Coli and/or L. Plantarum. This invention also provides a medical food for dietary management of anemia, thrombocytopenia, and neutropenia, which may be administered orally to humans in single dose as small as 20 mg/kg. The dosage of 100 mg/kg may be preferable. Inhibitory effects over GGTP may be enhanced by isoflavones, which are retained in isolated soy proteins. For the safety reasons, peptidoglycan from L. Plantarum may be preferable.

Disaccharide tetrapeptide is the part of the basic unit of the peptidoglycans of Gramm negative bacteria and L. Plantarum. The peptidoglycan is a single bag shaped highly cross-linked macromolecule that surrounds the bacterial cell membrane and provides rigidity. It consists of glycan (polysaccharide) backbone consisting of N-acetyl muramic acid and N-acetyl glucosamine with peptide side chains containing D- and L-amino acids and diaminopimelic acid. In the cell wall they are bound to teicholic acid and polisaccharide by a phosphate diester band. Basic unit was purified by Takase et. Al., (U.S. Pat. No. 4,545,932 1985). However, under certain preparation conditions, two aminosugars (N-acetyl-glucosamine) are linked to muramic acid. This bond remains basically intact after lysozyme hydrolysis.

A great deal of endotoxicity is caused by peptidoglycans derived from gram-positive bacteria. Its peptidoglycan is able to induce leukopenia and thrombocytopenia (Verhoef J. and Kalter E., Prog.Clin.Biol.Res. 1985; 189:101-113). Moreover, peptidoglycans and lipoteichoic acid can cause the induction of nitric oxide (NO) formation, shock, and organ failure in the rat (Kengatharan K, et al. J.Exp.Med., 1998;20:305-15). Disaccharide tetrapeptide, N-acetyl-glucosamine-muramyl-L-alanine-D-isoglutamine-meso-diaminopimelyl-L-alanine was shown to be cytotoxic in explanted hamster tracheal tissue and hamster tracheal epithelial cell culture (Luker K E., et al., Proc.Natl.Acad.Sci., 1993, 90: 2365-2369). This disaccharide terapeptide induces leukocytosis in cerebrospinal fluid, influx of protein into CSF, or brain edema, alone or in combination. Muramylpeptide carrying the diaminopimelyl-diaminopimelic acid cross-link specifically induced cytotoxic brain edema (Burroughs M, et al., J.Clin.Invest., 1993, 92:297-302). The structural analog N-acetyl-glucosaminyl-1,6-anhydro-N-acetylmuramyl-L-alanine, disaccharide dipeptide (GMTP), is responsible for synergizing with lipoteicholic acid thereby causing septic shock during gramm positive infection. Orally administered peptidoglycans enhance leukopenia by stimulating of the phagocytosis of splenetic neutrophils from mice. (Sasaki T., et al. J.Vet.Med.Sci. 1996, 58:85-6). In addition, peptidoglycans, well known tumor necrosis factor (TNF) alpha stimulators, promote rheumatoid arthritis inflammation (Simelyte E., et al., Infection and Immunity, 2000, 68:3535-3540).

It is well known fact that low molecular weight peptidoglycans, GMTP and GMDP (695 D), are mainly responsible for immunogenic effects. They are potent stimulators of TNF alpha production, which may be detrimental in patients with autoimmune conditions such as rheumatoid arthritis. Excessive level TNF alpha production could be extremely dangerous for patients with ARDS, stroke, and ischemic heart disease, who already have high preexisting production of TNF alpha. Moreover, combination of MDP and TNF alpha can cause proinflammatory effects, thus exaggerating chronic viral and bacterial infection.

The present invention has been completed on the basis of findings that inhibition over TNF alpha cytotoxic activity is based on direct cell protection by N-acetyl-glucosamine-N-acetyl-muramyl-L-alanine-meso-pimelic-acid-D-alanine without concurrent inhibition of TNF alpha production. Moreover, this production is enhanced two fold in comparison with GMDP. The cytoprotective effect is caused by the presence of two D-aminoacids, D-isoglutamine and D-alanine. Free NH2 and carboxyl group on side chain of diaminopimelic acid are responsible for enhanced stimulation of TNF alpha (Doktor W H A, et al., J.Biol.Chem., 1994, 269:4201-4206).

GMTP predominantly affects the phagocytes, enhances neutrophil ability to produce hydrogen peroxide. The monocyte stimulation leads to enhance synthesis of the TNF alpha and MG-CSF. This disaccharide tetrapeptide increases the functional activity of natural killer cells, which leads to the lysis of the target myeiloblastic cells K-562. Nevertheless, GMTP does not effect cellular immunity, which was demonstrated by absence of its effect on hypersensitivity of the delayed type. In addition, this disaccharide tetrapeptide does not affect the central cytokine of the cellular immunity-interferon-γ

In parallel, undesirable proinflammatory and immunogenic properties were avoided by adding soy isoflavones to 10 KD peptidoglycan fraction of L. Plantarum. They are weak cytoprotective agents, however, can acts synergistically with GMTP, which a basic unit of this fraction. Thus, such composition enhances the inhibition of TNF alpha cytotoxicity and reduces adverse side effects of TNF alpha stimulation. It provides exceptional safety and improved tolerance in people with autoimmune conditions. In addition, achieved GGTP inhibition leads to the repletion of glutathione, well-known TNF alpha inhibitor.

The present invention provides an oral inhibitor of TNF alpha is substantially free of low molecular weight peptidoglycans such as GMDP. The glucopeptide fraction of molecular weight of not higher than 3,000 D and not less than 300 D in the cell wall of gram positive bacteria may be purified by a known method of molecular weight fractionation of proteins or by ultrafiltration. The presented peptidoglycan for GGTP inhibition with concurrent inhibition of immunogenicity are significantly different from previous inventions related to probiotic peptidoglycans. All of them have presented probiotic peptidoglycans as the immunostimulators. Immunostimulatory properties were reported by Link and Pahud (U.S. Pat. No. 5,185,321, 1993) and by Yamazaki et al,(EP99104209, 1999). Moreover, Yamazaki et al. taught to increase immunogenicity by purifying low molecular weight fraction of 500-4000 peptidoglycans with increased percentage of very low molecular weight of 500 D peptidoglycans. Their main objective was to increase production of TNF alpha to the level comparable with the stimulation by muramyl dipeptide (MDP). More over, the structure of the peptidoglycans are different: there is lysine instead of diaminopimelic acid. The apoptosis modulating compositions and anemia, neutropenia, or thrombocytopenia medicament can be administered in a synergistic amount effective to teat or to prevent anemia, thrombocytopenia, or neutropenia.

In some embodiments of the invention, the peptidoglycans is administered to in the effective amount to treat or prevent anaplastic anemia caused by chronic viral infection. In this aspect of the invention, the GMTP containing peptidoglycans are administered to the subject to restore distroyed hematopoieses, caused by accelerated apoptosis. Anemia, neutropenia, or thrombocytopenia medicament is subsequently administered to the subject. This method is particularly useful in subjects who are particularly susceptible to bacterial or viral disease, such as children, immunocompromised subjects, and elderly subjects.

In other aspects, the method of the invention involves administering a high dose of an anemia, thrombocytopenia, or neutropenia medicament to a subject, without inducing side effects. Ordinarily, when an anemia, thrombocytopenia, or neutropenia medicament is administered in high doses, a variety of side effects can occur. As a result of these side effects, the anemia, thrombocytopenia, or neutropenia medicament is not administered in such high doses, no matter what therapeutic benefits are derived. It was discovered, according to the invention, that such high doses of anemia, thrombocytopenia, or neutropenia medicaments which ordinarily induce side effects can be administered without inducing the side effects as long as the subject also receives a peptidoglycan.

The peptidoglycan modulators of TNF alpha of the present invention have the following excellent features;

1. They are inhibitors of TNF alpha cytotoxicity originated from lactic acid bacteria L. Plantarum, which is used in the production of yogurt and fermented milk food and drinks.

2. They are the substances from natural origin which can exert cytoprotective effect at a dose much less than the known TNF alpha inhibitors produced by plants, sea weeds and microorganisms.

3. Since they are water soluble, they can be readily prepared in appropriate formulations. Latest feature is a real benefit for parenteral administration.

4. In composition with isolated soy proteins their effects over upregulated TNF alpha synthesis can be enhanced. Such food compositions also provide well-balanced daily source of the amino acids.

5. They are stimulators of the TNF alpha and GM-CSF synthesis.

As stated above, GGTP inhibition leads to the preservation of extracellular glutathione—powerful antioxidant with remarkable detoxification properties. In particular, the invention has application in alcohol detoxification, anesthetic recovery and in recovery or withdrawal from hypnotics, narcotics, sedatives or other drugs, especially in case of abuse. Treatment of withdrawal is a particular area where the invention has applicability.

The invention may have application in the prevention, treatment or management of toxicity caused directly or indirectly by one of the following compounds:

anesthetics, including: local anesthetics (such as cocaine, procaine, lidocaine, tetracaine, mepivacaine, bupivacaine and etidocaine, chlorprocaine), inhalational anaesthetics (such as methoxyflurane, halothane, enflurane, isoflurane and nitrous oxide); intravenous anesthetics (etomidate, benzodiazepines, and barbiturates);

opiods, including: heroin and morphine related opioids (such as hydromorphone, oxymorphone, levorphanol, codein, hydrocodon, oxycodone, nalorphine, naloxone, naltrexone, buprenorphine, butorphanol and nalbuphine);

sedatives and hypnotics, including barbiturates and benzodiazepines;

other drugs subjects to abuse, including cocaine and related drugs; nicotine and tobacco;

psychedelic drugs, which are hallucinogenics and/or psychotomimetics and/or psychotogenics;

ethanol and its metabolites.

The invention has applications in dealing with endogenous created toxins. Acetaldehyde, the primary metabolite of ethanol, is an example. Probiotic glucoproteins are useful for dietary management of the patients who accumulated endotoxins as a result of disease.

One endogenous toxin, which can often cause the problems, is bilirubin. High levels are known to results in jaundice, particularly in babies and patients with advanced hepatic metastases. Reduced liver function is also a characteristic feature of geriatric patients. In addition, in cancer patients, levels of drug such as analgetics and chemotherapeutics tend to build up in the body and this can lead to severe side effects. Yet, another endotoxins, free radicals, are generated during radiation and chemotherapy.

However, the applicant has been able to demonstrate that the level of metabolites such as bilirubin and iron in the blood of patients could be significantly reduced when patients are fed with probiotic glucoproteins.

Because of their exceptional inhibitory activity over TNF alpha, GMTP containing peptidoglycans are extremely valuable food for combating leukocytopenia and thrombocytopenia, which are common toxic side effects of radiation and chemotherapy in cancer patients. Precisely how leukocytopenia and thrombocytopenia are treated and prevented remains to be shown. Though, it is well established fact that the inhibitors of TNF alpha cytotoxicity, such as anti TNF alpha antibodies, an agent used to treat ulceratus colitis, prevents myelosuppresion in cancer patients treated with melphalan (Gupta V., et al. Cancer Chemother. Pharmacol. 1995;36:13-9).

From the above it can be seen that the invention also relates to a method for the prevention, treatment and management of bone morrow damage mediated by TNF alpha. In addition, the invention relates to nutrition for reducing damage after ribovarine treatment of hepatitis C.

Feeding with peptidoglycans of L. Plantarum is particularly effective in those patients who have increased risk of anaplastic anemia caused by chronic viral infection. Patients with severe thrombocytopenia after chemotherapy also can benefit from from proposed medical food by improving both platelets and neutrophil count.

Because of this specific TNF alpha inhibition, probiotic peptidoglycans of L. Plantarum are valuable for all conditions with upregulated TNF alpha synthesis. It seems that they are extremely useful for treating patients with anemia and leucopenia caused by sepsis.

Yet, among another indications is anemia caused by autoimmune diseases such as rheumatoid arthritis. The present inventor has carried out intensive research in order to develop safer agents, which can exhibit profound desensitizing effect without significant immunosupression. Consequently, he has found out that the decreasing percentage of low molecular weight peptidoglycans by ultrafiltration of ingredients less than 10000 D and higher than 30000 D creates glucoproteins composition which clinically does not lead to the symptoms of overproduction of TNF alpha, thereby is absolutely safe for people with rheumatoid arthritis. The present invention provides a medical food, which can desensitize T lymphocytes without any immunosuppression. The method for preparing medical food according to the present invention will now be explained. The GMP compositions to be used in the present invention may be obtained from a variety of Gram negative bacteria or gram positive bacteria L. Plantarum following known methods. The amino acid sequence and sugar composition of the complexes will be defined by a species of bacterium.

L. Plantarum is cultured via the culture conditions suitable for the microbiological properties of the species, to collect the cultured bacterial cells. These may be cultured in the culture medium routinely used for lactobacillus, for example, Rogosa medium, but complex medium using soy protein broth or distiller's soluble, etc. as nitrogen source may be also used. Peptones, yeast extracts, and glucose are most preferable ingredients of culture medium. The fermentation methods may follow the routine methods for lactobacilli. Routine methods for bacteria degradation such as ultrasound, temperature (hot water), and enzyme hydrolysis may be employed. Lysozyme hydrolysis is preferable one.

Routine methods for purifications of peptidoglycan complexes can be employed. More specifically, hydrolysate obtained by aforementioned methods, is applied to anion-exchange column to remove lysozyme and high-molecular nuclear acids. Further, protease and nuclease can be used for degradation of the remaining proteins and nuclear acids, respectively. Hydrophobic chromatography may be used to remove enzymes by passing them through a column with resin. Glucoprotein composition may be fractioned by gel chromatography.

Yet, nanofiltration by using 100 D, 200, or 300 D membranes and ultrafiltration by using membrane with cut off range of 3000 D-500000 D are considered most preferable method for purifications of the peptidoglycan compositions. More specifically, 50000 D polyethersulfone membrane may be used to filtrate nuclear acids and high molecular weight proteins from aforementioned lysozyme hydrolysite. Then, 1000 D membrane may be employed to eliminate salts and water from the composition. This high molecular fraction is indicated for anemia treatment in patients with autoimmune diseases, when immunogenic, proinflammatory peptidoglycans with low molecular weight may cause severe side effects. glucoproteins composition which clinically does not lead to the symptoms of overproduction of TNF alpha, thereby is absolutely safe for people with rheumatoid arthritis. The present invention provides a medical food, which can desensitize T lymphocytes without any immunosuppression. The method for preparing medical food according to the present invention will now be explained. The GMTP compositions to be used in the present invention may be obtained from a variety of Gram negative bacteria or gram positive bacteria L. Plantarum following known methods. The amino acid sequence and sugar composition of the complexes will be defined by a species of bacterium.

L. Plantarum is cultured via the culture conditions suitable for the microbiological properties of the species, to collect the cultured bacterial cells. These may be cultured in the culture medium routinely used for lactobacillus, for example, Rogosa medium, but complex medium using soy protein broth or distiller's soluble, etc. as nitrogen source may be also used. Peptones, yeast extracts, and glucose are most preferable ingredients of culture medium. The fermentation methods may follow the routine methods for lactobacilli. Routine methods for bacteria degradation such as ultrasound, temperature (hot water), and enzyme hydrolysis may be employed. Lysozyme hydrolysis is preferable one.

Routine methods for purifications of peptidoglycan complexes can be employed. More specifically, hydrolysate obtained by aforementioned methods, is applied to anion-exchange column to remove lysozyme and high-molecular nuclear acids. Further, protease and nuclease can be used for degradation of the remaining proteins and nuclear acids, respectively. Hydrophobic chromatography may be used to remove enzymes by passing them through a column with resin. Glucoprotein composition may be fractioned by gel chromatography.

Yet, nanofiltration by using 100 D, 200, or 300 D membranes and ultrafiltration by using membrane with cut off range of 3000 D-500000 D are considered most preferable method for purifications of the peptidoglycan compositions. More specifically, 50000 D polyethersulfone membrane may be used to filtrate nuclear acids and high molecular weight proteins from aforementioned lysozyme hydrolysite. Then, 1000 D membrane may be employed to eliminate salts and water from the composition. This high molecular fraction is indicated for anemia treatment in patients with autoimmune diseases, when immunogenic, proinflammatory peptidoglycans with low molecular weight may cause severe side effects.

Similarly, fractions, which compose of different percentage of low molecular weight glucopeptides, can be obtained by using 100 D reverse osmosis membrane and 3000 D or 10000 D polyethersulfone membranes. 1000 D membranes are used for filtrating low molecular pyrogenic muramyl peptides and glucomuramyl peptides, respectively, as well as salts and acetic acid. Employing 10000 D or 30000 D membranes can regulate percentage of high molecular weight peptidoglycans. Fractions obtained by aforementioned ultrafiltration are especially effective for inhibition of TNF alpha cytotoxicity in patients with anemia caused by chronic viral infection.

A fraction, which contains up 88% GMTP can be obtained by selective isolation of this tetrapeptide from peptidoglycans of gram negative bacteria such as E. Coli, B. Thyphimirium also suitable for this purpose.

A prefered peptidoglycan compositions can be obtained by mixing with isolated soy proteins N-acetyl-glucosamine. The amount of the probiotic glucoproteins of the total weight of a composition on dry basis is preferably more than 10 weight percent.

Preferred amounts of N-acetyl-glucosamine as weight percent shall be in the range of from about 10 to 30 percent, for example such as 20 weight percent.

Accordingly, weight ratio of isolated soy proteins is preferably more 1.0, for example 1.15.

Water processed soy proteins retaining a natural level of isoflavones are most preferable for mixing with probiotic glucoproteins.

Alternatively, the present invention provides a composition wherein probiotic and soy aminoacids serve as a daily source of protein.

The proposed composition can be served as a powder mixed with milk, orange juice or other beverage of choice.

### DESCRIPTION OF THE PREFERRED EMBODIMENTS

Specific production embodiments are presented hereinafter.

### EXAMPLE 1

### Isolation of Glucopeptides from Lactobacillus Plantarum

1. Biomass Preparation

15 kg of wet biomass was supplied by Chr. Hansen Corp., (Milwaukee, Wis.) Biomass was separated from the rest of the feeding media by washing with distilled water three times. Wet biomass was rinsed twice by 15 L of distilled water using centrifuge Backman JM-6 at 3900 rpm/min until supernatant liquid becomes colorless.

2. Hydrolysis.

3 kg moist biomass was resespended in 20 L H₂O and boiled for 15 min. After that, it was deluted in 30 L H₂O+NaHCO₃ (to achieve ₚH=6.0) and added 30 g of lyzosyme(Canadian Inovatech, Inc., Vancouver, Canada). Hydrolysis was done for 48 hours at 54° C. Then, 500 mll food grade vinigar was added to achieve pH=4.0 and was centrifiged on Beckman JM-6 g at 4000 rpm for 5 hours.

3. First Ultrafiltration.

Catridge with the membrane capable of retaining compounds with molecular weight less than 3000 D and with S=0.09M² at speed 2.5 L/h (Millipore Corp, USA) were used. 8.8 L solution with retained nuclear acids, phospholipids, and lysozyme was wasted.

4. Second Nanofiltration.

Cartridge with Nanomax® membrane installed on Prolab System, (Millipore Corp., USA) was used for filtrating compounds with molecular weight less than 300 D (muramylpeptides, salts, acetic and lactic acids). Then, 5L of retained was freeze dried. Yield was 110 g of molecular peptodoglycans with the molecular weight in the range of 300-3000 D.

### EXAMPLE 3

### LDH Assay of TNF Alpha Cytotoxicity

Lactate dehydrogenase (LDH) is a stable cytosolic enzyme, product of a housekeeping gene that is released upon cell lysis. Released product is measured in rapid enzymatic assay, measuring the conversion of tetrazolium salt into a red formazan product. The reaction have two steps: LDH is producing NADH which is reducing in the next step the salt to a red product. LDH assay has been used for variety of applications with many different cell types for measurement of cell mediated cytotoxicity, mediated by chemicals or other agent as well as changes in the total cell number.

In our experiments we used LDH release assay (CytoTox 96. Promega) in order to asses the TNF induced cell death and consecutively the cytoprotection provided by certain compound to the TNF and FAS induced cytolysis. The supplier of the kit recommended the procedure we used.

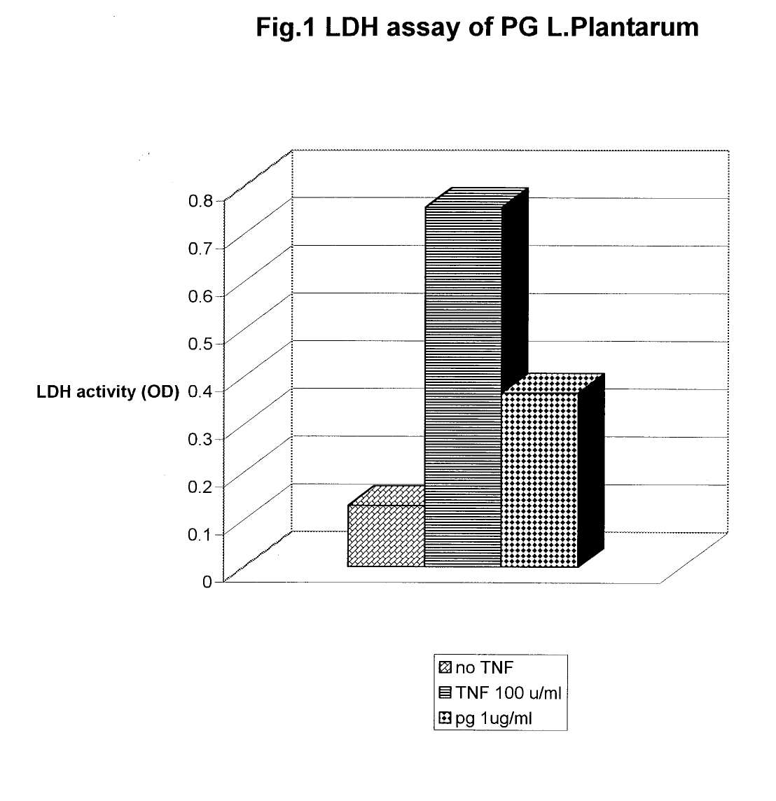

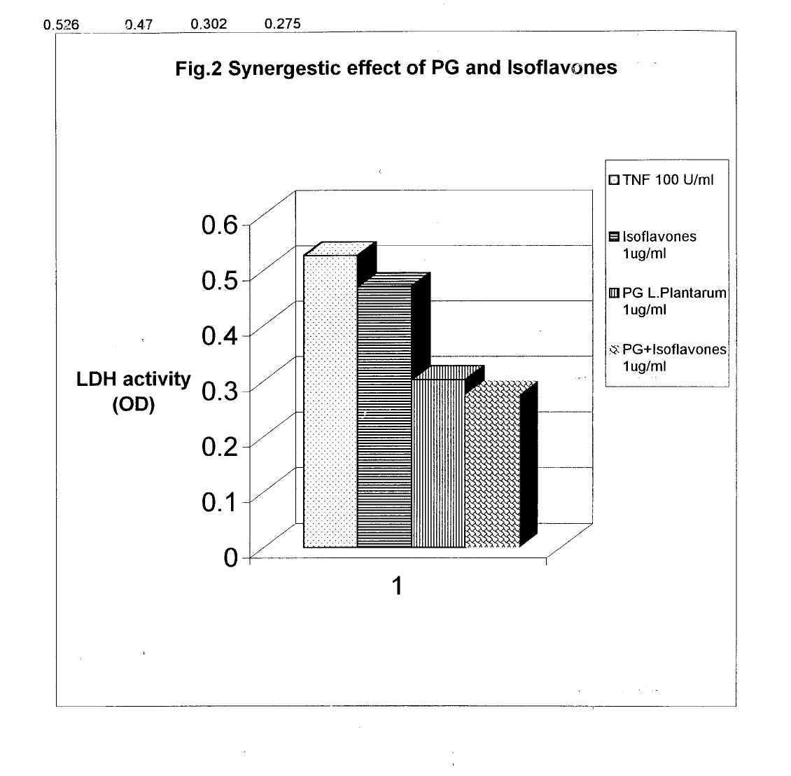

Briefly: A549 cells (human lung cancer) were seeded in six-well plates, and after 24 h (70% confluence) treated with 25 ug/ml cycloheximide (CHX) and either 100 U/ml human TNF (Beoringer) or. Twenty hours after the treatment 20 ul of the cultured supernatant was removed and tested for the LDH activity in 96 well plates in triplicate. Samples were assayed on an EL340 Microplate reader (Biotec Instruments, Inc) at 490-nm wavelength. FIG. 1 represents the the results of inhibition of LDH release by 1 μg/ml of peptidoglycan derived from L. Plantarum. FIG. 2 demonstrates the synergistic effect of soy isoflavones and peptidoglycans on LDH inhibition.

### EXAMPLE 3

### Effect Disaccharide Tetrapeptide on TNF Alpha Synthesis

Mononuclear cells of 7 healthy males were isolated on ficol-verografin gradient (density 1,077) and incubated for 2 days in CO2 incubator. Full culture media was RPMI-1640 with hentamicine 150 μg/ml, 10% inactivated embryonic weal serum, 15 mM of glutamine (‘Sigma”). PL-3 was added in concentrations of 100 μg/ml, 10 μg/ml, 1 μg/ml, and feeding media was added to the control wells. Cytokine content was defined by IFA with commercial kit Cytelisa (Cytimmune, USA).

TABLE 1
Effect of the different PL-3 and GMDP concentrations on TNF alpha
production by human mononuclear blood cells.

| | | GMDP | | | PL-3 | | |
|---|---|---|---|---|---|---|---|
| N | Control | 1 | 10 | 100 | 1 | 10 | 100 |
| 1 | 457 | 616 | 472 | 597 | 597 | 878 | 1117 |
| 2 | 123 | 287 | 221 | 250 | 202 | 652 | 730 |
| 3 | 399 | 516 | 512 | 390 | 328 | 771 | 815 |
| 4 | 466 | 593 | 639 | 749 | 530 | 634 | 611 |
| 5 | 103 | 86 | 80 | 224 | 755 | 807 | 1165 |
| 6 | 484 | 841 | 945 | 1107 | 541 | 351 | 426 |
| 7 | 103 | 103 | 166 | 276 | 172 | 259 | 264 |
| M | 305 | 435 | 434 | 513 | 446 | 621 | 733 |
| δ | 185 | 284 | 304 | 326 | 217 | 234 | 334 |

Thus, one can see that PL-3 causes a significant increase of TNF alpha synthesis by mononuclear cells. On the other hand, GMDP does not lead to the induction of this cytokine and the difference with control samples is not significant.

### EXAMPLE 4

### Effects GMTP on the Synthesis GM-CSF

The effect of PL-3 on the synthesis of GM-CSF was evaluated accordingly to the previous method.

It was noticed, that PL-3 and GMDP are the strong inductors of the GM-CSF synthesis (See Table 2). Both compounds cause statistically significant increase of GM-CSF production in comparison with its random synthesis (a control). PL-3 is more potent inductor. In the dosage of 10 mkg/ml and 100 mkg/ml it induces the cytokine synthesis almost two times more (statistically significant difference with p=0.029 and 0.038) than GMDP.

TABLE 2
The effect of GMTP and GMDP on the GM-CSF production
by the human mononuclear cells.

| | | GMDP | | Pl-3 | |
|---|---|---|---|---|---|
| Number | Control | 10 | 100 | 10 | 100 |
| 1 | 266 | 579 | 581 | 740 | 713 |
| 2 | 413 | 729 | 735 | 920 | 1132 |
| 3 | 266 | 778 | 773 | 1052 | 1248 |
| 4 | 423 | 941 | 983 | 1109 | 1483 |
| 5 | 522 | 555 | 584 | 752 | 887 |
| 6 | 402 | 543 | 609 | 740 | 1059 |
| 7 | 0 | 427 | 944 | 1115 | 2524 |
| M± | 617 ± 90.2 | 668.2 ± 167.9 | 744.1 ± 155 | 975 ± 282.5 | 1288 ± 598 |
| P< | — | 0.007 | 0.0007 | 0.00001 | 0.00001 |

### EXAMPLE 5

### Effect of GMTP on Apoptosis of the Human Mononuclear Cells

The longevity of the mononuclear leukocytes was evaluated by the incorporation of propidium iodide in the dosage of 1.25 μg/ml within 30 min at 37° C. The results were obtained with the flow cytometry (FACSCalibur, Bekton Dickinson, data processing program, CellQuest). The cells were used after 24, 48, and 72 hours of the incubation in the full cultural media in CO2-incubators in the presence of the different doses of the compound: 1, 10, 100 μg/ml. GMDP was used for the comparison.

The cell longevity was unchanged after 24 hours treatment with both GMDP and GMTP. The weak protective effect was observed after 48 hours of incubation. This effect became stronger after 72 hours of the incubation. There was a twofold reduction of the percentage of dead cells.

TABLE 3
Effect of GMTP on cell longivity.

| | |
|---|---|
| PL-3 | GMDP |

| Number | Control | 1 | 10 | 100 | 1 | 10 | 100 |
|---|---|---|---|---|---|---|---|
| 1 | 28 | 16 | 16 | 19 | 16 | 17 | 15 |
| 2 | 41 | 18 | 21 | 16 | 17 | 23 | 18 |
| 3 | 40 | 18 | 17 | 19 | 18 | 21 | 24 |
| M | 36 | 18 | 18 | 18 | 17 | 20 | 19 |
| δ | 7.2 | 1.2 | 2.6 | 1.7 | 1 | 3.1 | 4.6 |

### EXAMPLE 5

### The Effect of GMTP on the Formation of the Active Forms of the Oxygen by Leukocytes of the Healthy Donors

This effect was evaluated by luminal dependent chemoluminiscence and cytofluorometry.

In the first case leukocytes were separated by gelatin and connected with luminal, human serum and added to the vials with the different concentration of the substance. The control vials have carried the feeding media. The evaluation of the active oxygen forms was done at 370° C. during 30 min in the luminometer 1251 (LKB Corp.) It is well known that hydrogen peroxide on cell membrane is registered by luminal depedent chemoluminiscence.

In the second case the leukocytes were added to dichlorofluoroscein diacetete-DXF-DA(Sigma). This substance is originally non fluorescence one. However, after the penetration into the cells it is turned into green fluorochrom in the presence of the hydrogen peroxide. The reaction was monitored in the flow cytometer FACSCalibur.

TABLE 4

| | | GMTP | | | GMDP | | |
|---|---|---|---|---|---|---|---|
| N donor Control | | 1 | 10 | 100 | 1 | 10 | 100 |
| 1 | 80.9 | 97.4 | 97.3 | 105.1 | 87.2 | 103.0 | 90.5 |
| 2 | 208.7 | 203.9 | 203.1 | 217.7 | 202.3 | 197.0 | 199.3 |
| 3 | 301.2 | 290.6 | 277.5 | 273.4 | 260.2 | 257.8 | 258.7 |
| 4 | 180.8 | 190.3 | 183.9 | 181.8 | 177.6 | 179.4 | 172.8 |
| 5 | 91.5 | 85.1 | 83.7 | 84.3 | 91.9 | 83.7 | 93.2 |
| M | 172.6 ± 90.6 | 173.4 ± 84.4 | 168.9 ± 80.1 | 172 ± 78.4 | 163.8 ± 74.1 | 164.8 ± 71.2 | 162.9 ± 71.9 |

The incubation of the leukocytes of the healthy donors with GMTP and GMDP did not change their ability to generate of the active oxygen forms. Cytofluometric analysis has demonstrated, that GMTP stimulated the active oxygen forms in comparison with the control data (p=0.08). GMDP does not posses this activity. The donor N4 has had an elevated original hydrogen peroxodide, however the oxygen explosion was noticed only at low 1 μg dose of PL-3. Unlike neutrophils, monocytes do not generate the active oxygen forms neither with PL-3, nor with GMDP.

TABLE 5
The effect of GMTP and GMDP on the formation of the active oxygen
forms by neutrophils.

| | | GMDP | | | PL-3 | | |
|---|---|---|---|---|---|---|---|
| N | Control | 1.0 μg/ml | 10 μg/ml | 100 μg/ml | 1 μg/ml | 10 μ/ml | 100 μg/ml |
| 1 | 18 | 17.5 | 17 | 16 | 16 | 15 | 19 |
| 2 | 18 | 17.5 | 15 | 38 | 18 | 18 | 31 |
| 3 | 19 | 19 | 33 | 20 | 22 | 19 | 47 |
| 4 | 35 | 42 | 42 | 34 | 57 | 41 | 41 |
| 5 | 14 | 24 | 21 | 24 | 19 | 19 | 39 |
| 6 | 17 | 23 | 13 | 14 | 38 | 17 | 17 |
| 7 | 13 | 14 | 9 | 14 | 17 | 14 | 20 |
| M ± δ | 22.1 ± 8.7 | 23.8 ± 11.5 | 23.7 ± 12 | 2.8 ± 9.7 | 25.9 ± 13.5 | 20.4 ± 8.3 | 29.6 ± 10.7 |

TABLE 6
The effect of GMTP and GMDP on the formation of active oxygen
forms by monocytes.

| N | Control | GMDP | | | | GMTP | |
|---|---|---|---|---|---|---|---|
| 1 | — | 1.0 | 10.0 | 100.0 | 1.0 | 10.0 | 100.0 |
| 2 | 19 | 12 | 19 | 19.5 | 18 | 18 | 0.6 |
| 3 | 21 | 18 | 15 | 34 | 16 | 17 | 26 |
| 4 | 15 | 14 | 26 | 16 | 18 | 15 | 31 |
| 5 | 20 | 27 | 25 | 14 | 31 | 29 | 22 |
| 6 | 12 | 16 | 14 | 15 | 15 | 14 | 18 |
| 7 | 16 | 23 | 14 | 12 | 29 | 18 | 13 |
| 8 | 14 | 16 | 11 | 16 | 50 | 14 | 25 |
| 9 | 32 | 54 | 16 | 34 | 25 | 13 | 20 |
| 10 | 56 | 15 | 70 | 102 | 25 | 32 | 42 |
| M | 22.8 | 21.7 | 23.3 | 29.2 | 25.2 | 18.9 | 23.8 |
| δ | 13.8 | 13.0 | 18.2 | 28.5 | 10.9 | 6.9 | 8.7 |

### EXAMPLE 6

### Effect GMTP on the Functional Activity of Natural Killers

Mononuclears of the 7 healthy donors were isolated on fecol density gradient, d=1.007 in the full culture media with different concentrations GMTP and GMDP. Cells were separated after 3 hours of the incubation at 37° C. in CO2 incubator. Their cytotoxic action on the target cells K-562 labeled with ³H-uridine was evaluated.

TABLE 7
The effect of GMTP and GMDP on the function of natural killer cells.

| | | Cytotoxicity (%) in the presence of | | | | | |
|---|---|---|---|---|---|---|---|
| | | the different doses (μg/ml) | | | | | |
| | | GMTP g/ml | | | GMDP g/ml | | |
| N | Control | 1 | 10 | 100 | 1 | 10 | 100 |
| 1 | 59 | 63 | 65 | 66 | | | |
| 2 | 51 | 48 | 57 | 54 | 47 | 47 | 48 |
| 3 | 32 | 45 | 55 | 56 | 51 | 34 | 33 |
| 4 | 47 | 56 | 38 | 55 | 49 | 48 | 48 |
| 5 | 32 | 41 | 44 | 49 | 38 | 45 | 46 |
| 6 | 41 | 37 | 43 | 51 | 44 | 40 | 44 |
| 7 | 44 | 55 | 53 | 49 | 50 | 53 | 50 |
| M | 43.7 | 49.2 | 50.7 | 54.2 | 46.5 | 44.5 | 44.8 |
| δ | 9.8 | 9.1 | 9.4 | 5.8 | 4.8 | 6.6 | 6.1 |

The experiments have demonstrated that PL-3 possesses the ability to increase the cytotoxic activity of the natural killer cells in the dosage of 100 μg/ml

### EXAMPLE 7

### Dietary Management of Anaplastic Anemia

The patient S. 18 years old, was complaining on significant fatigue and prolonged bleeding during her menses. Objectively, she has had hemorrhagic petechiae in the skin all over her body. Anaplastic anemia was diagnosed based on the results of bone marrow biopsy. Epstein-Barr virus was detected and believed to be a cause of bone marrow anaplasia. She was placed on high doses of prednizone 350 mg daily, neoral 200 mg daily, cyclosporine 200 mg daily, Neupogen®, and and erethpoietin. Her condition was steadily deteriorating regardless of this treatment. One year later she started taking peptidoglycan of L. Plantarum, prepared accordingly to the example. The average dose was 1 g per day. The blood CBC results are presented in the table 8.

TABLE 8
Effect of probiotic peptidoglycans on blood CBC in a patients
with anaplastic anemia.

| | Platelets, | WBC, | RBC | | | Neutrophils, |
|---|---|---|---|---|---|---|
| Date | Thou/cmm | thou/cmm | million/cmm | Hb, g/dl | Hematocrit | Abs.Aut. |
| Feb. 15, 2001 | 22000 (L) | 2100 (L) | 2.56 (L) | 7.9 (L) | 24.5 (L) | 1.1 (L) |
| Feb. 19, 2001 | 11800 (L) | 4100 (L) | 2.36 (L) | 7.79 (L) | 23.1 (L) | 2.7 |
| Feb. 22, 2001* | 20000 | 2300 (L) | 2.43 (L) | 8.2 (L) | 24.1 (L) | 1.2 (L) |
| Feb. 26, 2001 | 17800 | 4000 (L) | 2.25 (L) | 7.69 (L) | 22.5 (L) | 2.6 |
| Mar. 5, 2001 | 18700 | 2300 (L) | 2.33 (L) | 8.10 (L) | 23.8 (L) | 1.4 |
| Mar. 12, 2001 | 29200 | 2100 (L) | 2.61 (L) | 8.9 (L) | 27.4 (L) | 4.7 |
| Mar.3, 19 2001 | 19900 | 3700 (L) | 2.39 (L) | 8.48 (L) | 25.3 (L) | 2.5 |
| Mar.3, 26 2001 | 25000 | 3900 (L) | 2.40 (L) | 8.2 (L) | 25.4 (L) | 2.6 |
| M | 20489 | 3100 (L) | 2.13 (L) | 8.12 (L) | 24.3 (L) | 2.1 |
| Apr. 2, 2001 | 23000 | 4000 (L) | 2.46 (L) | 8.85 (L) | 26.5 (L) | 2.7 |
| Apr. 9, 2001 | 23000 | 4200 | 2.46 (L) | 8.92 (L) | 26.5 (L) | 2.8 |
| Apr. 16, 2001 | 22000 | 2000 (L) | 2.41 (L) | 8.75 (L) | 26.0 (L) | 0.9 |
| Apr. 23, 2001 | 28000 | 4700 | 2.66 (L) | 9.59 (L) | 29.0 (L) | 3.2 |
| Apr. 30, 2001 | 38000 | 5100 | 2.99 (L) | 10.2 (L) | 31.4 (L) | 3.7 |
| M | 26800 | 4000 (L) | 2.58 (L) | 9.27 (L) | 27.98 | 2.66 |
| May 09, 2001 | 33000 | 2800 (L) | 2.78 (L) | 10.2 (L) | 30.0 | 49.2 |
| May 21, 2001 | 42000 | 2400 (L) | 3.09 (L) | 10.8 (L) | 32.8 | 46 |
| Jun. 4, 2001 | 39000 | 2500 (L) | 3.06 (L) | 10.6 (L) | 32.0 | 48 |
| Jun. 18, 2001# | 50000 | 2200 (L) | 3.01 (L) | 10.5 (L) | 31.4 | 48 |
| M | 41000 | 2470 | 2.98 | 10.5 | 31.5 | 43.82 |
| Sep. 4, 2001& | 47000 | 3100 (L) | 2.89 (L) | 10.2 (L) | 30.5 (L) | 1.7 (L) |
| Oct. 2 2001 | 60000 | 3700 (L) | 3.17 (L) | 11.6 (L) | 33.4 (L) | 1.9 |
| Oct. 30 2001 | 61000 | 3500 (L) | 3.25 (L) | 11.4 (L) | 34.2 (L) | 2.0 |
| Dec. 3, 2001 | 68000 | 5200 | 3.36 (L) | 11.6 (L) | 35.3 (L) | 3.0 |
| Jan. 14, 2002 | 67000 | 4000 (L) | 3.48 (L) | 12 | 36.1 (L) | 2.0 |
| Feb. 25, 2002 | 75000 | 4700 | 3.65 (L) | 12.5 | 38.4 | 2.9 |

*first day on peptidoglycans of L. Plantarum, average dose of 1 g per day
#cyclosporin was reduced to 50 mg/day with concurrent reduction of steroids.
&She is off cyclosporine and steroids

One can see the steady rise in the count of platelets, WBC, hemoglobin, neutrophils, and hematocrit after starting peptidoglycans of L. Plantarum.

We claim:

1. A soy isoflavones and peptidoglycan compositions of L. Plantarum for treating anemia, thrombocytopenia, and leucopenia in humans and domestically useful animals with concurrent hemapoietic cell apoptosis, compromising administering a therapeutically effective amount of GM containing peptidoglycans.

2. A method of treating of anemia, thrombocytopenia, and leukopenia by feeding humans and domestically useful animals which have a disease state characterized by hemapoietic cell apoptosis comprising administering a therapeutically effective amount of N-acetyl-glucosamine-N-acetyl-muramyl-L-alanine, D-isoglutamine-meso-diaminopimelyl-D-alanine.

3. A composition of claim 1 where molecular weight of isolated peptidoglycans is in the range of 100 D-3000 D

4. A composition of claim 1 where molecular weight of isolated peptidoglycans is in the range of 300 D-50000 D

5. A composition of claim 1 where anemia is anaplastic.

6. A composition of claim 1 where anemia is caused by viral infection.

7. A composition of claim 1 where anemia, thrombocytopenia, and leucopenia are caused by chemotherapy of cancer.

8. A method of claim 2 where GMTP is derived by lysozyme hydrolysis of E. coli.

9. A method of claim 2 where GMTP is isolated by applying dialysis and ultrafiltration.

10. A method of claim 2 where anemia is caused by viral infection.

11. A method of claim 2 where anemia, thrombocytopenia, and leucopenia is caused by chemotherapy of cancer.

12. A composition of claim 1 where hepoietic cell apoptosis leads to myielodisplastic syndrome.

13. A method of claim 2 where disease state is myielodisplastic syndrome.

14. A method of claim 2 where endogenous GM-CSF synthesis is stimulated for treating anemia, thrombocytopenia, and leukopenia.

15. A composition of claim 1 where anemia, thrombocytopenia, and leukopenia is associated with rheumathoid arthritis

16. A method of claim 2 where GGTP is administered in combination with anemia medicament selected from the group of consisting of EPO, recombinant G-CSF, recombinant interleukin 11, ferrous iron, ferric iron, vitamin B12, vitamin B6, vitamin C, vitamin D, calcitriol, alphacalcidol, folate, androgen, and carnitine.

17. A method of claim 1 where peptidoglycan compositions are administered in combination with anemia medicament selected from the group of consisting of EPO, recombinant G-CSF, recombinant interleukin 11, ferrous iron, ferric iron, vitamin B12, vitamin B6, vitamin C, vitamin D, calcitriol, alphacalcidol, folate, androgen, and carnitine.

18. A method of claim 1 where peptidoglycan compositions are administered in combination with thrombocytopenia medicament selected from the group consisting of a glucocorticoid, recombinant thrombopoietin, recombinant megakaryocyte growth and development factor, lisophylline, recombinant IL-1, recombinant IL-3, recombinant IL-6, and recombinant IL-11.

19. A method of claim 2 where GGTP is administered in combination with the neutropenia medicament selected from the group consisting of glucocorticoid, recombinant G-CSF, recombinant GM-CSF, recombinant macrophage colony-stimulating factor, recombinant IL-1, recombinant IL-3, recombinant IL-6, immunoglobulin, androgens, recombinant IFN-gamma, small molecule G-CSF mimetics, G-CSF receptor antagonists, IL-3 receptor antagonist, uteroferrin.

20. A method of claim 1 where peptidoglycan compositions are administered in combination with the neutropenia medicament selected from the group consisting of glucocorticoid, recombinant G-CSF, recombinant GM-CSF, recombinant macrophage colony-stimulating factor, recombinant IL-1, recombinant IL-3, recombinant IL-6, immunoglobulin, androgens, recombinant IFN-gamma, small molecule G-CSF mimetics, G-CSF receptor antagonists, IL-3 receptor antagonist, uteroferrin.
